(19) United States
(12) Patent Application Publication
(10) Pub. No.: US 2024/0081774 A1
(43) Pub. Date: Mar. 14, 2024

(54) Fatigue Detection Device, Support System, And Fatigue Detection Method

(71) Applicant: SEIKO EPSON CORPORATION, Tokyo (JP)

(72) Inventors: Mio SASAKI, Shiojiri (JP); Kanechika KIYOSE, Matsumoto (JP)

(21) Appl. No.: 18/466,290

(22) Filed: Sep. 13, 2023

(30) Foreign Application Priority Data
Sep. 14, 2022 (JP) 2022-146271

(51) Int. Cl. A61B 8/08

(52) U.S. Cl. CPC A61B 8/0858

(57) ABSTRACT

A fatigue detection device includes an ultrasonic device configured to transmit an ultrasonic wave into a living body, then receive the ultrasonic wave reflected by a tissue in the living body to output a received signal, and at least one processor, wherein the processor obtains the received signal output from the ultrasonic device, and detects a fatigue state of the tissue from a change in the received signal when a load is applied to the living body.

The present application is based on, and claims priority from JP Application Serial Number 2022-146271, filed Sep. 14, 2022, the disclosure of which is hereby incorporated by reference herein in its entirety.

### BACKGROUND

### 1. Technical Field

The present disclosure relates to a fatigue detection device, a support system, and a fatigue detection method.

### 2. Related Art

In the past, when measuring a fatigue state of a muscle, blood is sampled from a living body to measure a level of lactic acid in the blood. However, in such an invasive method, it is unachievable for general users to easily measure the fatigue of a muscle.

Meanwhile, as a method of non-invasively measuring the fatigue state, there has been known a method of measuring fatigue information using a bioelectrical impedance analysis (see, e.g., JP-A-2021-23497 (Document 1)). In this device, a weak current is transmitted to and received from a living body using electrodes to measure the bioelectrical impedance, and then whether or not swollenness is getting resolved, or whether or not the swollenness has been resolved is evaluated based on a change in the bioelectrical impedance. Thus, it becomes possible to detect the fatigue due to the swollenness. Further, the biological information such as height, weight, age, and sex input by the user and the bioelectrical impedance thus measured are combined with each other to obtain body fat percentage, muscle mass, water amount, and so on of the user. Further, stiffness (fatigue) of the living body is evaluated based on the hardness of a body surface estimated from a body composition value or the hardness of a living body surface measured using a body surface hardness meter.

A fatigue detection device described in Document 1 measures the biological impedance using a non-invasive method, but is limited to what determines whether or not the swollenness has been resolved, namely the fatigue deriving from the swollenness. Further, when performing the evaluation of the stiffness, the body surface hardness meter is additionally required, and thus, the configuration is complicated.

### SUMMARY

A fatigue detection device according to a first aspect of the present disclosure includes an ultrasonic device configured to transmit an ultrasonic wave into a living body, then receive the ultrasonic wave reflected by a tissue in the living body to output a received signal, and at least one processor, wherein the processor obtains the received signal output from the ultrasonic device, and detects a fatigue state of the tissue from a change in the received signal when a load is applied to the living body.

A support system according to a second aspect of the present disclosure is a support system including the fatigue detection device described above, a fatigue information processor, and an annunciator, wherein the fatigue detection device outputs fatigue information including information related to a fatigue state to the fatigue information processor, the fatigue information processor determines a health state of a user based on the fatigue information, and the annunciator informs the user of information related to the health state determined by the fatigue information processor.

A fatigue detection method according to a third aspect of the present disclosure is a fatigue detection method of detecting a fatigue state of a tissue using an ultrasonic device configured to transmit an ultrasonic wave into a living body, and receive the ultrasonic wave reflected by the tissue in the living body, the method including outputting a received signal corresponding to the ultrasonic wave which is transmitted into the living body from the ultrasonic device, then reflected by the tissue, and then received by the ultrasonic device, and detecting a fatigue state of the tissue from a change in the received signal when a load is applied to the living body.

### BRIEF DESCRIPTION OF THE DRAWINGS

FIG. 1 is a block diagram showing a schematic configuration of a support system according to a first embodiment.

FIG. 2 is a perspective view schematically showing a schematic configuration of a health support device according to the first embodiment.

FIG. 3 is a diagram showing a wear example of the health support device according to the first embodiment.

FIG. 4 is a diagram showing another wear example of the health support device according to the first embodiment.

FIG. 5 is a diagram showing a configuration example of a surface of the health support device according to the first embodiment, wherein the surface adheres to a human body.

FIG. 6 is a diagram showing another configuration example of the surface of the health support device according to the first embodiment, wherein the surface adheres to the human body.

FIG. 7 is a diagram showing another configuration example of the surface of the health support device according to the first embodiment, wherein the surface adheres to the human body.

FIG. 8 is a plan view showing an example of an ultrasonic substrate constituting an ultrasonic device in the first embodiment.

FIG. 9 is a schematic cross-sectional view of the ultrasonic device in the first embodiment cut along the line B-B shown in FIG. 8.

FIG. 10 is a flowchart showing a fatigue detection method and a health support method using a support system 1 according to the first embodiment.

FIG. 11 is a block diagram showing a schematic configuration of a support system according to a second embodiment.

FIG. 12 is a flowchart showing a fatigue detection method and a health support method using the support system 1 according to the second embodiment.

FIG. 13 is a block diagram showing a schematic configuration of a support system according to a third embodiment.

FIG. 14 is a flowchart showing a fatigue detection method and a health support method using a support system LA according to the third embodiment.

### DESCRIPTION OF EXEMPLARY EMBODIMENTS

### First Embodiment

A first embodiment of the present disclosure will hereinafter be described.

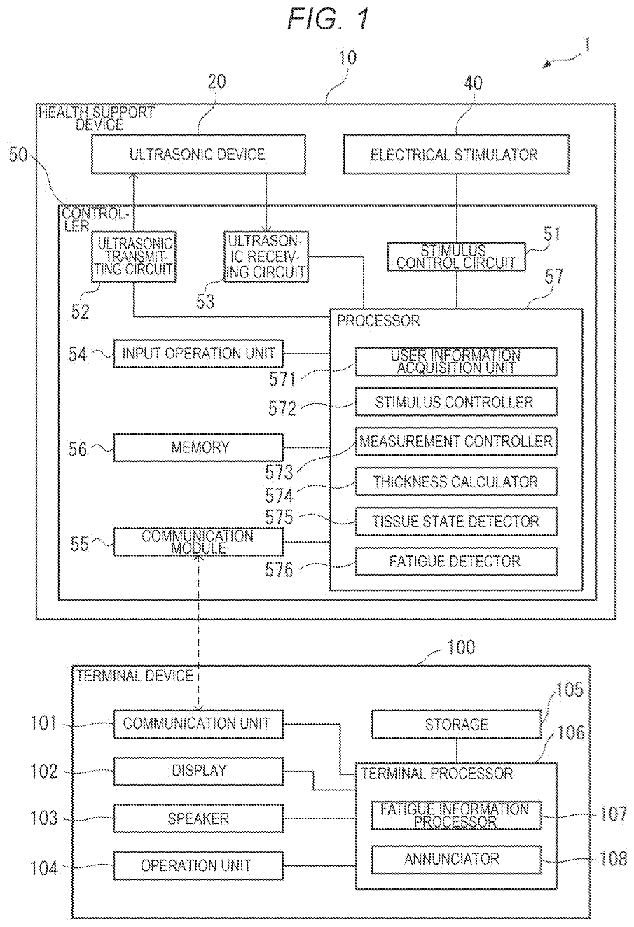

FIG. 1 is a block diagram showing the schematic configuration of a support system according to the present embodiment.

A support system 1 according to the present embodiment is provided with a health support device 10, and a terminal device 100 disposed so as to be able to communicate with the health support device 10.

The health support device 10 also functions as a fatigue detection device according to the present disclosure, and is worn by a living body (a human body in the present embodiment) to detect the fatigue of predetermined tissue of the living body.

Specifically, the health support device 10 performs ultrasonic measurement on the tissue in the human body to measure a change in thickness (tissue thickness) of the tissue, and then detects the fatigue based on the rate of the change in tissue thickness. Further, the health support device 10 transmits fatigue information related to muscle fatigue to the terminal device 100.

It should be noted that it is possible to illustrate an internal organ such as a muscle, a blood vessel, or a stomach as the tissue, and in the present embodiment, the description will hereinafter be presented illustrating a device for detecting the fatigue of the muscle. Specifically, in the present embodiment, the health support device 10 applies an electrical current to the muscle of a predetermined region in the human body to thereby apply a stimulus to the muscle to make the muscle perform a contractile motion, and measures a change in muscle thickness before and after the contractile motion to thereby detect the fatigue state.

The terminal device 100 receives the fatigue information transmitted from the health support device 10, then determines the health state based on the fatigue information, and then informs a user of the health state thus determined. For example, when using the fatigue information obtained by detecting the fatigue of the muscle, the terminal device 100 introduces an exercise heavier in load when there is no fatigue in the muscle, or announces a decrease in load or a rest in accordance with the condition of the muscle fatigue.

A variety of constituents constituting the support system 1 will hereinafter be described in detail.

Configuration of Health Support Device 10

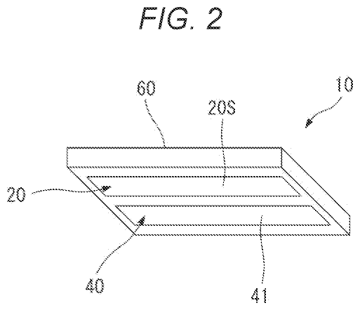

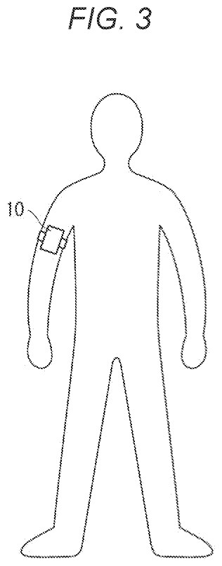

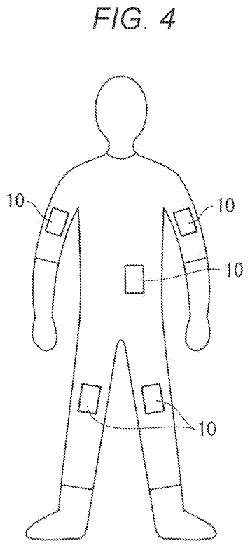

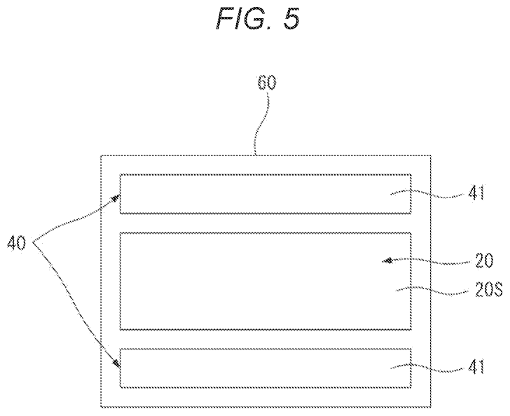

FIG. 2 is a perspective view schematically showing a schematic configuration of the health support device 10 according to the present embodiment. FIG. 3 and FIG. 4 are diagrams showing a wear example of the health support device 10. FIG. 5 through FIG. 7 are each a schematic diagram showing an example of a surface which is made to adhere to the human body in the health support device 10.

As shown in FIG. 1, the health support device 10 is configured including an ultrasonic device 20, an electrical stimulator 40, a controller 50, and a chassis 60 for housing the ultrasonic device 20, the electrical stimulator 40, and the controller 50.

In the present embodiment, the ultrasonic device 20, the electrical stimulator 40, and the controller 50 are housed in the chassis 60 having a thin box-like shape. It should be noted that there is illustrated here an example in which the ultrasonic device 20, the electrical stimulator 40, and the controller 50 are housed in the chassis 60, but it is possible to adopt a configuration in which, for example, the ultrasonic device 20 and the electrical stimulator 40 are arranged on a sheet member having flexibility, and the chassis 60 housing the controller 50 is disposed in a part of the sheet member.

As shown in FIG. 3 and FIG. 4, the health support device 10 according to the present embodiment can be attached to a desired position of the human body, and for example, an arm, a leg, and a stomach area can be illustrated.

As a wear method of the health support device 10 to the human body, the chassis 60 is made to adhere to the human body, and is then fixed with a medical tape or band as shown in, for example, FIG. 3. Alternatively, as shown in FIG. 4, it is possible to adopt a configuration of disposing the health support device 10 on a reverse surface (a surface at the human body side) of a contact type suit which adheres to the human body, and it is possible to dispose a plurality of the health support devices 10.

On the surface of the chassis 60 which is made to adhere to the human body, there are exposed an ultrasonic transmitting/receiving surface 20S of the ultrasonic device 20 and an electric pad 41 of the electrical stimulator 40. When attaching the health support device 10 to the human body, an intermediary layer such as gel or water is made to intervene between the ultrasonic transmitting/receiving surface 20S and the human body, and between the electric pad 41 and the human body, and the ultrasonic transmitting/receiving surface 20S and the human body, and the electric pad 41 and the human body are made to adhere to each other so as not to include a bubble.

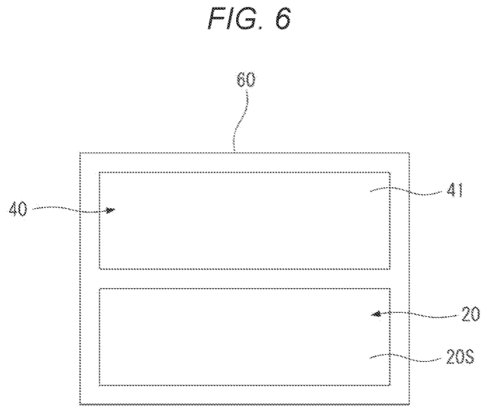

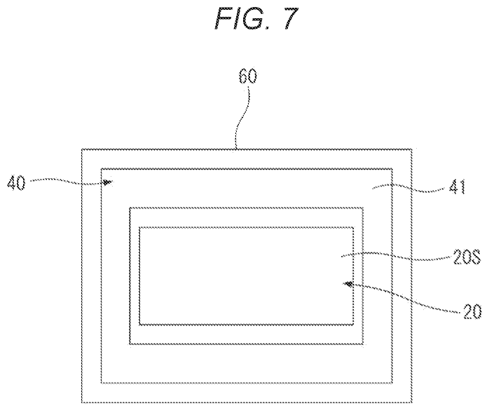

As shown in FIG. 5 through FIG. 7, in the present embodiment, the ultrasonic transmitting/receiving surface 20S and the electric pad 41 are arranged so as to be adjacent to each other. A relationship between the arrangement positions of the ultrasonic transmitting/receiving surface 20S and the electric pad 41 is not particularly limited. For example, as shown in FIG. 5, it is possible to dispose a pair of electric pads 41 across the ultrasonic transmitting/receiving surface 20S. Alternatively, as shown in FIG. 6, it is possible to arrange a single ultrasonic transmitting/receiving surface 20S and a single electric pad 41 so as to be adjacent to each other. Alternatively, it is possible to adopt a configuration in which the electric pad 41 is arranged so as to surround the periphery of the ultrasonic transmitting/receiving surface 20S as shown in FIG. 7. The relative positional relationship between the ultrasonic transmitting/receiving surface 20S and the electric pad 41 described above can arbitrarily be set in accordance with a posting position of the health support device 10 to the human body.

Due to such a configuration, it becomes possible to measure the state of the tissue in the human body to which the electric stimulus is applied by the electric pad 41 using the ultrasonic device 20.

Configuration of Ultrasonic Device 20

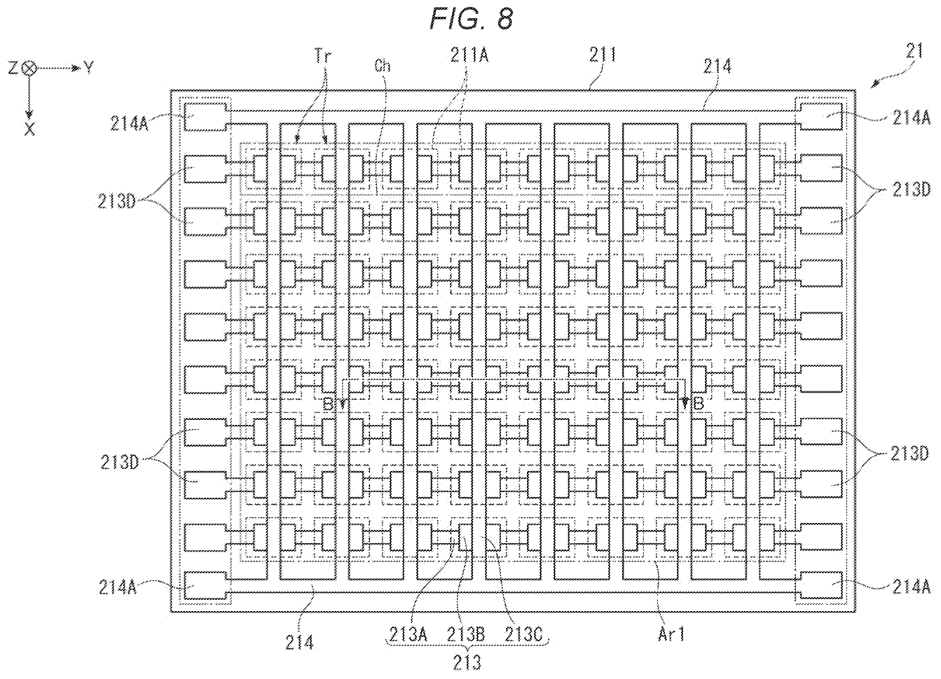

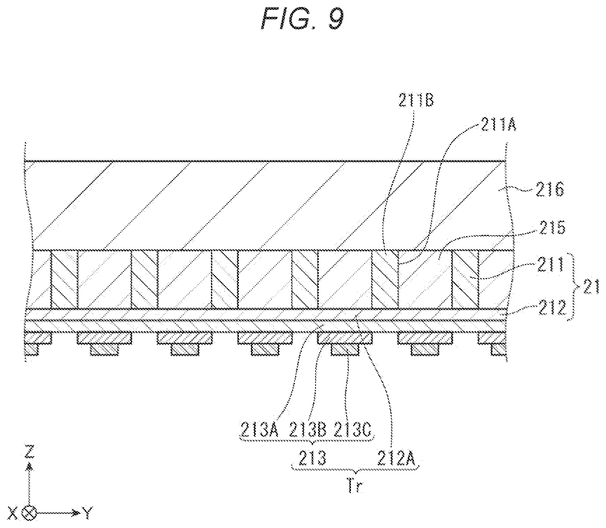

The ultrasonic device 20 is configured including an ultrasonic substrate 21 on which a plurality of ultrasonic transducers Tr (see FIG. 8, FIG. 9) for performing transmission and reception of an ultrasonic wave is arranged in an array.

FIG. 8 is a plan view showing an example of the ultrasonic substrate 21 in the present embodiment, and FIG. 9 is a schematic cross-sectional view of the ultrasonic substrate 21 cut along the line B-B shown in FIG. 8.

On the ultrasonic substrate 21, there is arranged the plurality of ultrasonic transducers Tr in a two-dimensional array along an X direction and a Y direction. Here, a direction perpendicular to the X direction and the Y direction is defined as a Z direction, and the Z direction (+Z) corresponds to a transmission direction in the present disclosure in which the ultrasonic wave is transmitted.

In the present embodiment, 1-CH (channel) transmitting/receiving column Ch is constituted by the plurality of ultrasonic transducers Tr arranged in the Y direction. Further, a plurality of the 1-CH transmitting/receiving columns Ch arranged side by side along the X direction constitutes an ultrasonic array Arl having a one-dimensional array structure. In the present embodiment, each of the transmitting/receiving columns Ch constitutes an ultrasonic element according to the present disclosure.

It should be noted that in FIG. 8, the number of the ultrasonic transducers Tr arranged is reduced for the sake of convenience of explanation, but in reality, a larger number of ultrasonic transducers Tr can be arranged.

As shown in FIG. 9, the ultrasonic substrate 21 is configured including an element substrate 211, a vibrating plate 212 disposed on the element substrate 211, and piezoelectric elements 213 disposed on the vibrating plate 212.

The element substrate 211 is formed of a semiconductor substrate made of, for example, Si. The element substrate 211 is provided with substrate opening parts 211A corresponding to the respective ultrasonic transducers Tr. In the present embodiment, each of the substrate opening parts 211A is a through hole penetrating the element substrate 211 in the substrate thickness direction (the Z direction), and the vibrating plate 212 is disposed at the −Z side of the through hole.

Further, at a side (+Z) of the substrate opening part 211A at which the vibrating plate 212 is not disposed, the substrate opening part 211A is filled with an acoustic layer 215 having the acoustic impedance approximate to that of the living body. As the acoustic layer 215, there can be used a resin material such as silicone.

Further, at the +Z side of the element substrate 211, there can be disposed a protective layer 216 formed of a resin material such as silicone, and there can be disposed an acoustic lens.

The vibrating plate 212 is formed of, for example, a stacked body of SiO₂ and ZrO₂, and is disposed so as to cover the entire area of a surface at one side of the element substrate 211. Specifically, the vibrating plate 212 is supported by partition walls 211B constituting the substrate opening parts 211A, and closes the −Z side of the substrate opening parts 211A. The thickness dimension of the vibrating plate 212 is made sufficiently small with respect to that of the element substrate 211.

The piezoelectric elements 213 are disposed respectively on parts of the vibrating plate 212 closing the respective substrate opening parts 211A. The piezoelectric elements 213 are each formed of, for example, a stacked body obtained by stacking a lower electrode 213A, a piezoelectric film 213B, and an upper electrode 213C from the vibrating plate 212 toward the −Z side.

Here, the part of the vibrating plate 212 closing the substrate opening part 211A constitutes a vibrating part 212A, and the vibrating part 212A and the piezoelectric element 213 constitute one ultrasonic transducer Tr.

In such an ultrasonic transducer Tr, by applying a rectangular-wave voltage (a drive voltage) having a predetermined frequency between the lower electrode 213A and the upper electrode 213C, the piezoelectric film 213B is deflected to vibrate the vibrating part 212A to transmit the ultrasonic wave toward the +Z side. Further, when the vibrating part 212A is vibrated by the ultrasonic wave (a reflected wave) reflected by the living body, an electrical potential difference occurs between an upper part and a lower part of the piezoelectric film 213B. Thus, by detecting the electrical potential difference occurring between the lower electrode 213A and the upper electrode 213C, it becomes possible to detect the ultrasonic wave received.

As shown in FIG. 8, in the present embodiment, the lower electrode 213A is formed along the Y direction to have a linear shape, and couples the plurality of ultrasonic transducers Tr constituting the 1-CH transmitting/receiving column Ch to each other. Drive terminals 213D thereof are electrically coupled to the controller 50 via, for example, a flexible printed board.

Further, the upper electrode 213C is formed along the X direction to form a linear shape, and couples the ultrasonic transducers Tr arranged in the X direction to each other. Further, the end parts at the ±X sides of the upper electrode 213C are connected to a common electrode line 214. The common electrode line 214 connects the upper electrodes 213C arranged along the Y direction to each other, and common terminals 214A are disposed in end portions of the common electrode line 214. The common terminals 214A thereof are electrically coupled to the controller 50 via, for example, a flexible printed board.

Further, in the present embodiment, there is shown an example in which the ultrasonic arrays Arl each having the one-dimensional array structure are formed in the ultrasonic substrate 21, but it is possible to form ultrasonic array having a two-dimensional array structure as a configuration of individually driving the ultrasonic transducers Tr arranged in the Y direction and the ultrasonic transducers Tr arranged in the X direction, respectively.

It is possible to adopt a configuration in which such an ultrasonic substrate 21 as described above is further provided with a reinforcing plate in order to increase the substrate strength. When disposing the reinforcing plate, the reinforcing plate is disposed on a surface at an opposite side to the element substrate 211 of the vibrating plate 212. On this occasion, it is preferable to dispose a spacer (a bonding layer) in an area other than the vibrating part 212A, and bond the reinforcing plate via the spacer so that a vibration space for each of the vibrating parts 212A can be kept.

Configuration of Electrical Stimulator 40

The electrical stimulator 40 is a device for applying an electrical stimulus to a tissue in the human body to cause a contractile motion. In the present embodiment, as an example thereof, there is shown an example of applying an electrical stimulus to a muscle.

Although a description of a specific configuration of the electrical stimulator 40 is omitted, there is provided a configuration of an electrical stimulator for rehabilitation, or a configuration of a skeletal muscle training device using an electrical stimulus. Specifically, the electrical stimulator 40 is provided with the electric pad 41 which is made to adhere to the human body via the intermediary layer such as gel or water, and applies an electrical current having a predetermined frequency and a predetermined level to the inside of the human body from the electric pad 41 under the control of the controller 50.

It should be noted that in the present embodiment, there is described an example of making the muscle in the human body perform the contractile motion using the electrical stimulator 40, but the electrical stimulator 40 is not essential, and can be eliminated. In other words, the health support device 10 according to the present embodiment is for measuring a change in muscle thickness before and after, or during an exercise of the muscle as a measurement target tissue to thereby detect the fatigue, and it is sufficient for the user him- or herself to behave so that the tissue moves. For example, it is possible for the user to perform an exercise by him- or herself using exercise equipment such as a dumbbell to make the muscle perform the contractile motion, and in this case, it is possible to make configuration of the electrical stimulator 40 unnecessary. Further, when the measurement target tissue is a stomach, when the user wears the health support device 10 in the vicinity of the stomach in the stomach area around eating, it is possible to measure the state of the stomach during the eating and before and after the eating. Alternatively, when the measurement target is a blood vessel, since the blood vessel is physically active in a pulsed manner without an intension of the user, it is possible to make the configuration of the electrical stimulator 40 unnecessary.

Configuration of Controller 50

The controller 50 controls the ultrasonic device 20 and the electrical stimulator 40. Further, the controller 50 detects a change in tissue to be a target using the ultrasonic device 20 before using, during using, and after using the electrical stimulator 40, and then detects the fatigue state of that tissue from the change in the tissue. For example, in the present embodiment, the controller 50 controls the electrical stimulator 40 to apply periodic electrical stimuli to the muscle to thereby cause periodic muscle contractions in the muscle. Further, the controller 50 controls the ultrasonic device 20 to measure each of the thickness of the muscle before the muscle contraction and the thickness of the muscle during the muscle contraction, and at the same time, calculate a difference (an amount of change) in the muscle thickness between before and after the muscle contraction, or a ratio (a ratio of change) in the muscle thickness between before and after the muscle contraction. By continuously using the electrical stimulator 40, the muscle gradually fatigues, and the amount of the change and the ratio of the change during the muscle contraction also decrease gradually.

Therefore, it becomes possible for the controller 50 to determine the fatigue state of the muscle by monitoring the amount of the change or the ratio of the change.

Specifically, as shown in FIG. 1, the controller 50 is configured including a stimulus control circuit 51, an ultrasonic transmitting circuit 52, an ultrasonic receiving circuit 53, an input operation unit 54, a communication module 55, a memory 56, a single processor 57 or a plurality of processors 57, and so on.

The stimulus control circuit 51 applies a voltage to the electric pad 41 to make an electrical current flow through the human body. Further, the stimulus control circuit 51 arbitrarily controls an amount and a frequency of the electrical current to be applied to the human body under the control of the processor 57.

The ultrasonic transmitting circuit 52 outputs a drive signal to drive each of the transmitting/receiving columns Ch based on an instruction from the processor 57 to transmit the ultrasonic wave. The ultrasonic transmitting circuit 52 can be disposed for each of the transmitting/receiving columns Ch, or it is possible to adopt a configuration in which a single ultrasonic transmitting circuit 52 and the plurality of transmitting/receiving columns Ch are coupled to each other with a switch circuit, and the transmitting/receiving column Ch for outputting the drive signal can be selected with the switch circuit.

The ultrasonic receiving circuit 53 processes a received signal output from the transmitting/receiving columns Ch, and then outputs the received signal thus processed to the processor 57. The ultrasonic receiving circuit 53 can be disposed for each of the transmitting/receiving columns Ch, or it is possible for a single ultrasonic receiving circuit 53 and the plurality of transmitting/receiving columns Ch to be coupled to each other via a switch circuit.

The ultrasonic transmitting circuit 53 obtains a signal value of the received signal output from the transmitting/receiving columns Ch at predetermined sampling intervals, and the received signal including a change in signal value along the time line is input to the processor 57.

The time for obtaining the received signal by the ultrasonic receiving circuit 53 is a predetermined time set in advance from a transmission timing of transmitting the ultrasonic wave from the transmitting/receiving column Ch using the ultrasonic transmitting circuit 52, and is hereafter referred to as a determination time. The determination time can arbitrarily be set in accordance with a depth range in which the ultrasonic measurement is performed.

The input operation unit 54 receives an input operation by the user. As the input operation unit 54, it is possible to adopt a configuration provided with, for example, operation buttons or operation knobs, and it is possible to adopt a touch panel configured integrally with a display separately disposed.

It should be noted that it is also possible to adopt a configuration in which the input operation unit 54 is not disposed, and a variety of input operations from the terminal device 100 coupled to the controller 50 so as to be able to communicate with each other are input.

The communication module 55 communicates with the terminal device 100. The communication system of the communication module 55 is not particularly limited. For example, it is possible to adopt a wireless communication system such as infrared communication or Bluetooth (a registered trademark), it is possible to communicate with the terminal device 100 via an access point of a wireless LAN, or it is possible to adopt a configuration of communicating with the terminal device 100 from that access point via the Internet.

The memory 56 is a recording medium on which a variety of programs including a support program for performing the calculation of the change in the tissue thickness and the detection of the fatigue state using the ultrasonic device 20, and a variety of types of data to be used in the variety of programs are recorded.

Specifically, in the memory 56, there are stored user information related to the user to be a measurement target, a boundary determination model for determining a boundary of a desired tissue from a measurement result of the ultrasonic wave, a fatigue determination program for determining the fatigue state from the change in the desired tissue based on the measurement result of the ultrasonic wave, and so on. It should be noted that the user information is information related to the body of the user such as the age, the sex, the height, the weight of the user.

The processor 57 retrieves and then executes the variety of programs stored in the memory 56 to thereby perform a variety of types of arithmetic processing, and functions as a user information acquisition unit 571, a stimulus controller 572, a measurement controller 573, a thickness calculator 574, a tissue state detector 575, a fatigue detector 576, and so on.

The user information acquisition unit 571 obtains body information of the user via the input operation unit 54. It should be noted that it is possible to obtain the body information from the terminal device 100 which is coupled to the controller 50 so as to be able to communicate with each other, via the communication module 55. Further, the user information acquisition unit 571 stores the body information thus obtained in the memory 56 so as to be associated with a user ID for identifying the user.

The stimulus controller 572 outputs a stimulus command to the stimulus control circuit 51, wherein the stimulus command instructs the stimulus control circuit 51 to apply an electrical current from the electric pad 41. The stimulus command includes a current value and a frequency of the electrical current to be applied to the human body, and the stimulus control circuit 51 applies the voltage corresponding to the current value based on the stimulus command to the electric pad 41 with the frequency based on the stimulus command to apply the electrical current to the human body.

The measurement controller 573 outputs a transmission command of the ultrasonic wave to the ultrasonic transmitting circuit 52 to thereby transmit the ultrasonic wave from each of the transmitting/receiving columns Ch, and then obtains the received signal input from each of the transmitting/receiving columns Ch via the ultrasonic receiving circuit 53. The measurement controller 573 sequentially drives, for example, the plurality of transmitting/receiving columns Ch independently of each other to obtain the received signal from each of the transmitting/receiving columns Ch.

The thickness calculator 574 calculates the thickness of the target tissue in the human body based on the received signal obtained from each of the transmitting/receiving columns Ch.

Specifically, the thickness calculator 574 identifies the boundary of the tissue in the human body from the respective received signals obtained from the transmitting/receiving columns Ch, and then calculates the tissue thickness from the boundary thus identified.

As an identification of the boundary, it is possible to use, for example, a boundary determination model generated by machine learning. For example, in the received signal, there is included feature information such as time when a signal value takes a peak, a peak signal value, a variation (a standard deviation) of the signal value within a predetermined range from the peak, an average value or a central value of the signal values of all of the received signals. The peak of the received signal has a high possibility of corresponding to the boundary of any of tissues in the human body, and the peak positions become boundary candidates of the tissue. Therefore, by performing input to a boundary determination model which defines the feature information, the body information, and the boundary candidates as input, and defines flag information (boundary information) representing whether or not the boundary candidate is the boundary of the desired tissue as output, it is possible to determine whether or not each of the boundary candidates is the boundary of the tissue to be the measurement target.

Further, when the boundaries are determined from the boundary candidates using the boundary determination model, the thickness calculator 574 calculates the thickness (the tissue thickness) of the desired tissue from these boundaries.

The tissue state detector 575 detects the change ratio of the tissue thickness as the change in tissue.

For example, by applying an external stimulus such as an electrical stimulus to the muscle, the muscle contracts, and the muscle thickness changes. Therefore, the tissue state detector 575 detects a ratio (the change ratio) between the muscle thickness when the stimulus is applied to the muscle and the muscle thickness after the stimulus is removed.

It should be noted that as the change in tissue, it is possible to adopt a change amount as a difference between the muscle thickness when the stimulus is applied to the muscle and the muscle thickness after the stimulus is removed. It should be noted that the change amount of the muscle thickness due to the muscle contraction becomes large in the user large in muscle amount, and the change amount of the muscle thickness due to the muscle contraction becomes small in the user small in muscle amount. Therefore, there occurs a necessity of setting a threshold value used when determining the fatigue of the muscle for each of the users. In contrast, in the case of the change ratio of the muscle thickness, the influence of such an individual difference as described above is small, and it is easy to set the threshold value used when determining the fatigue of the muscle.

The fatigue detector 576 determines the fatigue state based on the change ratio of the tissue thickness thus detected.

For example, when the change ratio of the tissue thickness is no higher than the threshold value set in advance, a determination as “fatigue” is made. Further, it is possible for the fatigue detector 576 to determine which one of the plurality of fatigue levels the fatigue belongs based on the change ratio of the tissue thickness. For example, it is possible to determine “extreme fatigue” when the change ratio is lower than a first threshold value, determine “fatigue” when the change ratio is no lower than the first threshold value and lower than a second threshold value, determine “slight fatigue” when the change ratio is no lower than the second threshold value and lower than a third threshold value, and determine “no fatigue” when the change ratio is no lower than the third threshold value. These threshold values can arbitrarily be changed based on the user information. For example, when the user recorded on the user information is in his or her twenties, it is possible to set the threshold value lower compared to when the user is in his or her fifties.

Configuration of Terminal Device 100

The terminal device 100 can be formed of a general computer such as a smartphone, a tablet terminal, or a personal computer.

By installing a health support application dedicated when using the support system 1 in the terminal device 100, and the terminal device 100 executing the health support application, the terminal device 100 communicates with the health support device 10 to perform the health support of the user.

Specifically, the terminal device 100 has a configuration of a general computer, and is provided with a communication unit 101, a display 102, a speaker 103, an operation unit 104, a storage 105, a processor, and so on as shown in FIG. 1. In order to distinguish the processor provided to the terminal device 100 from the processor 57 provided to the health support device 10, the processor provided to the terminal device 100 is hereafter referred to as a terminal processor 106.

The communication unit 101 communicates with the health support device 10. As described above, the communication system with the health support device 10 is not particularly limited, and infrared communication, Bluetooth, wireless LAN, and so on can be illustrated. Further, the communication unit 101 communicates with other external equipment via the Internet.

The display 102 displays a variety of types of information as images under the control by the terminal processor 106.

The speaker 103 outputs a variety of types of information as sounds under the control by the terminal processor 106.

The operation unit 104 receives an input operation by the user. The configuration as the operation unit 104 is not particularly limited, and it is possible to adopt, for example, a touch panel integrated with the display 102, or it is possible to adopt a keyboard, a mouse, or the like.

In the storage 105, there are stored a variety of programs including the health support application described above, and a variety of types of data. Further, in the storage 105, there is stored the user information related to the user who uses the health support device 10.

The terminal processor 106 retrieves and then executes the variety of programs stored in the storage 105 to thereby function as a fatigue information processor 107 and an annunciator 108.

The fatigue information processor 107 receives the fatigue information related to the fatigue state of the tissue from the health support device 10, and then performs a variety of types of processing corresponding to the fatigue state to determine the health state of the user.

The annunciator 108 announces the health state determined by the fatigue information processor 107 or a variety of types of information corresponding to the health state. As the annunciation method by the annunciator 108, it is possible to make the display 102 display the annunciation information as an image, or it is possible to output the annunciation information from the speaker 103 as a sound.

For example, in the present embodiment, the health support device 10 also functions as a muscle training device for applying the electrical stimulus to the muscle to thereby contract the muscle. In this case, the fatigue information processor 107 receives the fatigue information of the muscle to be the target, and determines a recommended load of the muscle training, a duration of the training, and a rest in accordance with the fatigue information, and then the annunciator 108 announces the recommended load, the duration, the rest, and so on thus determined.

Fatigue Detection Method and Support Method by Support System 1

Then, a fatigue detection method and a health support method using such a support system 1 as described above will be described.

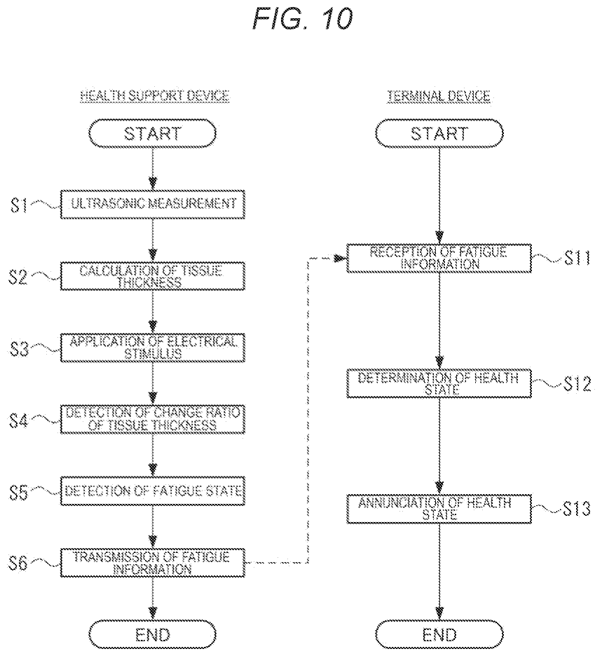

FIG. 10 is a flowchart showing the fatigue detection method and the health support method using the support system 1 according to the present embodiment.

Here, there is described an example in which the health support device 10 according to the present embodiment applies the electrical stimulus to the muscle with the electrical stimulator 40 to perform the training of the muscle.

The user first wears the health support device 10 in a desired region of the human body when performing the muscle training. For example, the user applies gel to the region to which the health support device 10 is attached, and then fixes the health support device 10 so that the ultrasonic transmitting/receiving surface 20S of the ultrasonic device 20 and the electric pad 41 of the electrical stimulator 40 adhere to the human body via the gel.

Subsequently, the user turns ON the power of the health support device 10. Further, the user operates the terminal device 100 to execute the health support application. It should be noted that it is assumed that user ID for identifying the user who uses the health support device 10 in advance are set and input to the health support device 10 in advance, and the user information related to the user is registered.

Thus, the measurement controller 573 of the health support device 10 controls the ultrasonic device 20 to perform (step S1) ultrasonic measurement, and the thickness calculator 574 calculates (step S2) the thickness (the tissue thickness) of the muscle as the measurement target tissue.

In the ultrasonic measurement by the ultrasonic device 20 in the step S1, the ultrasonic wave with a predetermined frequency is transmitted from the ultrasonic device 20 into the human body, then the ultrasonic wave is received in a predetermined period set in advance, and the received signals output from the ultrasonic device 20 are obtained with a predetermined sampling period. It is sufficient for the predetermined period to be set no shorter than the time necessary for the ultrasonic wave transmitted into the human body to be reflected by the desired tissue and then return to the ultrasonic device 20. Thus, the received signal obtained by a single ultrasonic measurement includes a first peak value due to the ultrasonic wave reflected by a boundary between a subcutaneous tissue and the muscle, and a second peak value due to the ultrasonic wave reflected by a boundary between the muscle and an underlying tissue (an internal organ, a bone, or the like). It should be noted that it is possible to obtain the muscle thickness by multiplying a time difference between a first timing at which the first peak value is obtained and a second timing at which the second peak value is obtained by the speed of the ultrasonic wave propagating through the human body.

In the way hereinabove described, the thickness of the muscle to which the external stimulus has not been applied is measured.

Subsequently, the stimulus controller 572 controls the electrical stimulator 40 to apply (step S3) the electrical current to the muscle in the human body from the electric pad 41. It should be noted that the current value and the frequency of the electrical current applied in the step S3 are set in, for example, initial values input in advance by the user. Thus, the electrical stimuli are continuously applied to the muscle with the frequency thus set to repeat the muscle contraction, and thus, the training of the muscle is started.

It should be noted that the processing in the step S1 and the step S2 is hereafter performed continuously with a constant period. Specifically, the ultrasonic measurement in the step S1 described above is performed with a constant period, and every time the ultrasonic measurement is performed, the calculation of the muscle thickness in the step S2 is performed. The period of the ultrasonic measurement is shorter than a period of the application of the electrical stimulus performed in the step S3, and the ultrasonic measurement is performed at least at the timing when the electrical current is applied from the electrical stimulator 40 and the timing when no electrical current is applied from the electrical stimulator 40. Thus, it is possible to obtain a transition of the muscle thickness along the time line.

Further, the tissue state detector 575 detects (step S4) the change ratio of the muscle thickness (the tissue thickness) from the muscle thickness values continuously obtained with the constant period. In the present embodiment, the electrical stimulus is periodically applied to the muscle in the step S3, and the contraction of the muscle due to the electrical stimulus and an expansion (a restoration motion) of the muscle due to a stoppage of the electrical stimulus are repeated. Therefore, when paying attention to the transition of the muscle thickness, a local maximum value and a local minimum value alternately appear. The tissue state detector 575 calculates a ratio between the local maximum value and the local minimum value which appears following that local maximum value, namely the change ratio of the muscle thickness.

Then, the fatigue detector 576 detects (step S5) the fatigue state of the muscle based on the change ratio of the muscle detected in the step S4.

In the present embodiment, the fatigue detector 576 compares the change ratio of the muscle thickness thus detected and a predetermined threshold value with each other to detect the fatigue state. For example, as described above, it is possible to detect the “fatigue” state when the change ratio is no higher than the threshold value thus set. Alternatively, it is possible to detect an “extreme fatigue” state when the change ratio is lower than the first threshold value, detect a “fatigue” state when the change ratio is no lower than the first threshold value and lower than the second threshold value, detect a “slight fatigue” state when the change ratio is no lower than the second threshold value and lower than the third threshold value, and detect a “no fatigue” state when the change ratio is no lower than the third threshold value.

Then, the fatigue detector 576 transmits (step S6) the fatigue information including the fatigue state thus detected to the terminal device 100.

When the fatigue information processor 107 of the terminal device 100 receives (step S11) the fatigue information from the health support device 10, the fatigue information processor 107 performs (step S12) the determination of the health state corresponding to the fatigue state.

For example, in the present embodiment, the fatigue information processor 107 generates recommended training information related to the output of the electrical stimulator 40 in accordance with the fatigue state.

For example, when it is received that the muscle is in the “fatigue” state as the fatigue information, the recommended training information of introducing a stoppage of the training is generated, and when it is received that the muscle is not in the “fatigue” state, the recommended training information of introducing a continuation of the training is generated.

Alternatively, when more detailed states are detected as the fatigue state, it is possible to generate more detailed recommended training information in accordance with the states. It is possible to generate the recommended training information of introducing, for example, the stoppage of the training in the case of the “extreme fatigue” state, the reduction of the training in the case of the “fatigue” state, the continuation of the training in the case of the “slight fatigue” state, and an enhancement of the training in the case of the “no fatigue” state.

Subsequently, the annunciator 108 performs (step S13) an annunciation of the health state thus determined.

For example, in the present embodiment, the annunciator 108 makes the recommended training information thus generated be displayed on the display 102, or be output from the speaker 103 as a sound.

### Functions and Advantages of Present Embodiment

The health support device 10 according to the present embodiment is provided with the ultrasonic device 20, and the single processor 57 or the plurality of processors 57. The ultrasonic device 20 transmits the ultrasonic wave into the human body (the living body), and then receives the ultrasonic wave reflected by the inside of the human body to output the received signal. The processor 57 obtains the received signal output from the ultrasonic device 20 to detect the fatigue state of the tissue from the change in the received signal when the load is applied to the human body.

Thus, in the health support device 10 according to the present embodiment, it is possible to detect the fatigue state of the tissue using the non-invasive method, and it is possible even for an ordinary user who does not have medical expertise to easily confirm the fatigue state of him- or herself. Further, for example, when detecting the change in value of a level of lactic acid in the blood or the change in biological impedance deriving from the swollenness to determine the fatigue, the detection accuracy of the fatigue state is far from high, and is large in individual difference since the state of the muscle is not directly detected, and the fatigue determination is based on other elements deriving from the fatigue. In contrast, as in the present embodiment, in the ultrasonic measurement, it is possible to directly detect the thickness and the action of the tissue, and thus, it is possible to make the detection accuracy of the fatigue state higher.

In the health support device 10 according to the present embodiment, the processor 57 functions as the tissue state detector 575 and the fatigue detector 576. The tissue state detector 575 detects the change in tissue from the tissue thickness calculated based on the received signal. The fatigue detector 576 detects the fatigue state of the tissue based on the change in the tissue thus detected.

Thus, it is possible to directly detect the state of the tissue from the received signal as the measurement data obtained by the ultrasonic measurement, and thus, it is possible to increase the detection accuracy of the fatigue state.

Here, the tissue state detector 575 detects the change ratio of the thickness of the tissue when the load is applied to the tissue as the change in the tissue.

It is possible to detect a difference (a change amount) between the tissue thickness when the external stimulus is applied to the tissue, and the tissue thickness when the tissue is released from that external stimulus, but such a change amount of the tissue thickness becomes large in individual difference. For example, when detecting the fatigue of the muscle, the amount of the change between when applying the external stimulus and when being released from the external stimulus becomes large in a person who is large in original muscle mass, and becomes small in a person who is small in muscle mass by contraries. In contrast, when detecting the change ratio between the tissue thickness when the external stimulus is applied to the tissue and the tissue thickness when the tissue is released from that external stimulus, it is possible to make the difference due to such an individual difference as described above small, and thus, it is possible to increase the detection accuracy of the fatigue detection.

Further, in the support system 1 according to the present embodiment, there are provided such a health support device 10 as described above and the terminal device 100. The health support device 10 transmits the fatigue information related to the fatigue state thus detected to the terminal device 100. The terminal device 100 functions as the fatigue information processor 107 and the annunciator 108. The fatigue information processor 107 determines the health state of the user based on the fatigue information. The annunciator 108 informs the user of the information related to the health state determined by the fatigue information processor.

Thus, it is possible to announce a variety of health states related to the fatigue state of the user, and it is possible to perform the health support of the user in good condition.

### Second Embodiment

Then, a second embodiment will be described.

In the first embodiment, there is described the example in which the fatigue detector 576 detects the change ratio of the tissue thickness calculated based on the received signal to detect the fatigue state based on whether or not the change ratio is lower than a predetermined threshold value, or to detect the fatigue state based on which one of a plurality of ranges set in advance the change ratio of the tissue thickness belongs to.

In contrast, in the present embodiment, the fatigue state is detected using the received signal obtained by the ultrasonic device 20 and a machine learning model (a fatigue detection model) for determining the fatigue state, in which the present embodiment is different from the first embodiment described above.

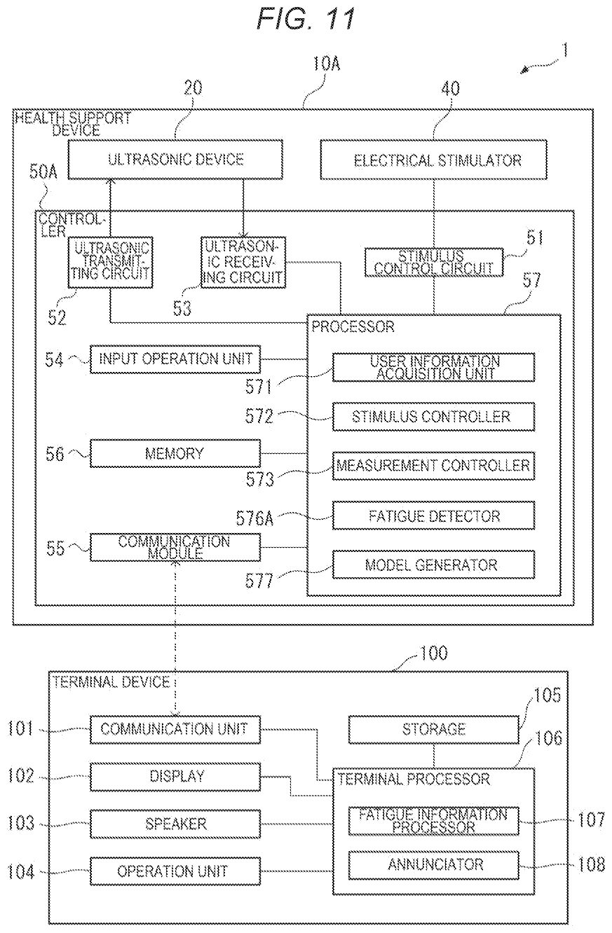

FIG. 11 is a block diagram showing a schematic configuration of a health support device 10A in the second embodiment.

In the following description, the constituents having already been described are denoted by the same reference symbols, and the description thereof will be omitted or simplified.

As shown in FIG. 11, in the present embodiment, the health support device 10A is provided with the ultrasonic device 20, the electrical stimulator 40, and a controller 50A.

Similarly to the first embodiment, the controller 50A in the present embodiment is provided with the stimulus control circuit 51, the ultrasonic transmitting circuit 52, the ultrasonic receiving circuit 53, the input operation unit 54, the communication module 55, the memory 56, the single processor 57 or the plurality of processors 57, and so on.

Further, in the present embodiment, on the memory 56, there is recorded the fatigue detection model. This fatigue detection model is a machine learning model taking at least the received signal as the input, and the fatigue state as the output. It should be noted that as the data to be input, the user information, and stimulus drive information representing a current value, a frequency, and so on of an electrical current output from the electrical stimulator 40 can be included in addition to the received signal.

This fatigue detection model is created by accumulating the received signals obtained by continuously driving the electrical stimulator 40 in the state in which the health support device 10A is attached to a predetermined region of the user, and periodically performing the transmitting/receiving processing of the ultrasonic wave by the ultrasonic device 20.

Further, in the present embodiment, the processor 57 retrieves and then executes the variety of programs stored in the memory 56 to thereby function as the user information acquisition unit 571, the stimulus controller 572, the measurement controller 573, a fatigue detector 576A, a model generator 577, and so on.

The model generator 577 creates the fatigue detection model using machine learning.

For example, the model generator 577 continuously applies the electrical stimuli to the muscle to perform a measurement set a plurality of times, and then accumulates the received signals of the measurement sets in the memory 56, wherein the ultrasonic measurement is periodically performed a plurality of times in the measurement set.

When periodically applying the electrical stimulus to the muscle, the contraction and the expansion of the muscle thickness are alternately repeated. As described above, the received signal obtained by a single ultrasonic measurement by the ultrasonic device 20 includes the first peak value due to the ultrasonic wave reflected by the boundary between the subcutaneous tissue and the muscle, the second peak value due to the ultrasonic wave reflected by the boundary between the muscle and the underlying tissue (an internal organ, a bone, or the like), the first timing at which the first peak value is obtained, and the second timing at which the second peak value is obtained. Therefore, by continuously performing the ultrasonic measurement with a predetermined period, it is possible to obtain the received signal representing the change in the muscle thickness when continuously applying the electrical stimulus to the muscle.

In the present embodiment, by accumulating a plurality of such received signals, it becomes possible to analyze the change in muscle thickness of each of the users. For example, when analyzing the transition of the change ratio of the muscle thickness along the time line when applying the electrical stimulus, the change ratio of the muscle thickness is high at a starting point of the muscle training, the change ratio gradually decreases, and finally converges with a substantially constant change ratio. The fact that increase and decrease of the change ratio along the time line substantially vanish means that the muscle fatigues. Thus, the change ratio when the increase and the decrease of the change ratio along the time line becomes substantially constant can be determined as a state in which the muscle extremely fatigues.

The model generator 577 generates the fatigue detection model for each of the users, and updates the fatigue detection model stored in the memory 56 using such received signals as described above as teacher data. It should be noted that as the teacher data, it is possible to further include the body information of the user, stimulus data including the current value and the frequency of the electrical current output from the electrical stimulator 40, and so on. The generation and the update of the fatigue detection model by the model generator 577 can be performed with a predetermined period such as every week, or can also be performed every time a predetermined number of received signals are accumulated.

Further, in the present embodiment, the fatigue detector 576A inputs the received signals to the fatigue detection model generated by the model generator 577 to thereby obtain the fatigue state output from the fatigue detection model. It should be noted that as the data to be input to the fatigue detection model, it is possible to further add the user information and the stimulus data.

Then, a fatigue detection method and a health support method using the support system according to the present embodiment will be described.

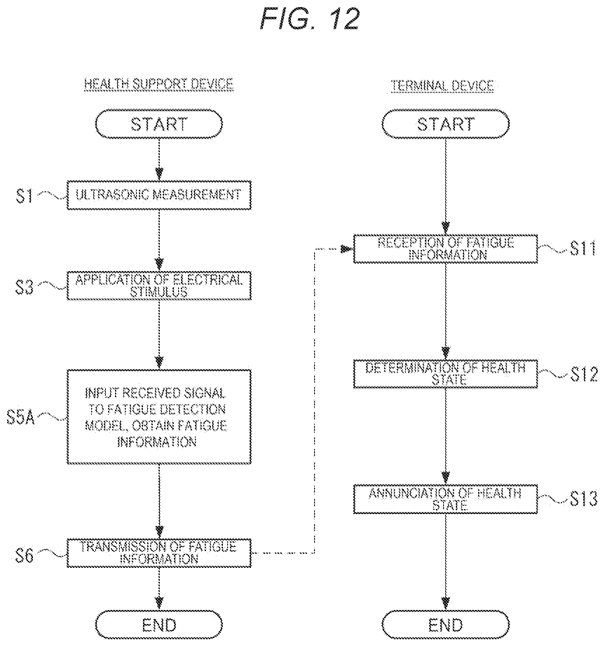

FIG. 12 is a flowchart showing the fatigue detection method using the support system 1 according to the present embodiment.

In the present embodiment, similarly to the first embodiment, the step S1 is performed to perform the ultrasonic measurement by the ultrasonic device 20, and thus, the received signal is obtained. Subsequently, in the present embodiment, the step S3 is performed to apply the electrical stimulus to the muscle.

Similarly to the first embodiment, the ultrasonic measurement in the step S1 and the application of the electrical stimulus to the muscle in the step S3 are each performed with a constant period. The period of the ultrasonic measurement in the step S1 is shorter than the period of the application of the electrical stimulus in the step S3, and the ultrasonic measurement is performed at least at the timing (a first time) when the electrical current is applied from the electrical stimulator 40 and the timing (a second time) when no electrical current is applied from the electrical stimulator 40.

Subsequently, the fatigue detector 576A inputs (step S5A) the received signals thus obtained to the fatigue detection model. The fatigue detector 576A inputs at least a first received signal obtained by the ultrasonic measurement at the first time when the electrical current is applied from the electrical stimulator 40, and a second received signal obtained by the ultrasonic measurement at the second time when no electrical current is applied from the electrical stimulator 40 to the fatigue detection model. Thus, the fatigue information related to the fatigue state of the muscle is output from the fatigue detection model.

Subsequently, the step S6 is performed, and the fatigue detector 576A transmits the fatigue information to the terminal device 100. Thereafter, the processing in the step S11 through the step S13 similarly to the first embodiment is performed.

### Functions and Advantages of Present Embodiment

In the health support device 10A according to the present embodiment, the processor 57 also functions as the model generator 577. The model generator 577 accumulates the received signals, and then performs the machine learning using the received signals thus accumulated as the teacher data to thereby generate the fatigue detection model taking the received signals as the input data, and the fatigue information representing the fatigue state as the output.

Further, the fatigue detector 576A inputs the received signals including the first received signal as an ultrasonic measurement result when (the first time) applying the electrical stimulus to the muscle by the electrical stimulator 40, and the second received signal as an ultrasonic measurement result when (the second time) no stimulus is applied to the muscle to the fatigue detection model, and then obtains the fatigue information.

In such a configuration, it is possible to make the processing of calculating the muscle thickness and the calculation processing of the change ratio of the muscle thickness unnecessary, and it is possible to more easily detect the fatigue state with a simpler configuration.

Further, since the fatigue detection model is generated based on the result of the ultrasonic measurement in each of the users, it is possible to generate the fatigue detection model personalized in accordance with a feature of each of the users, and thus, it is possible to further increase the fatigue detection accuracy.

### Third Embodiment

Then, a third embodiment will be described.

In the second embodiment described above, there is described the example in which the fatigue is detected using the fatigue detection model personalized for each of the users.

In contrast, in the third embodiment, there is described an example in which the fatigue detection model is generated based on the ultrasonic measurement results of a plurality of users.

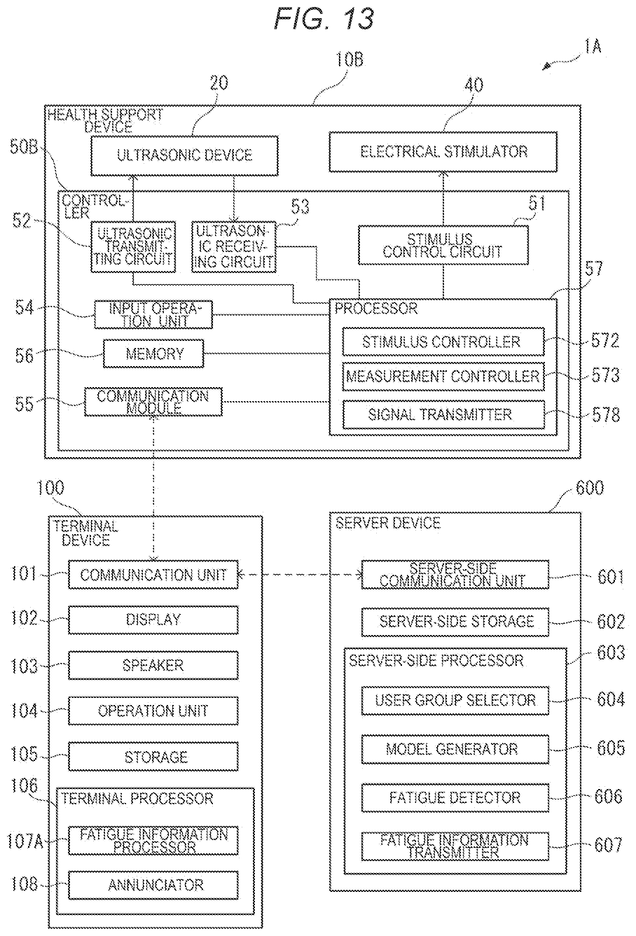

FIG. 13 is a schematic diagram showing an outline of a support system LA according to the third embodiment.

The support system according to the present embodiment is provided with a health support device 10B, the terminal device 100 corresponding to the health support device 10B, and a server device 600.

Similarly to the first embodiment and the second embodiment, the health support device 10B is provided with the stimulus control circuit 51, the ultrasonic transmitting circuit 52, the ultrasonic receiving circuit 53, the input operation unit 54, the communication module 55, the memory 56, and the single processor 57 or the plurality of processors 57.

Further, in the present embodiment, the processor 57 retrieves and then executes the variety of programs stored in the memory 56 to thereby function as the stimulus controller 572, the measurement controller 573, and a signal transmitter 578.

Specifically, when the health support device 10B in the present embodiment performs the ultrasonic measurement using the ultrasonic device 20 under the control of the measurement controller 573, the signal transmitter 578 transmits the received signal as the result of the ultrasonic measurement to the terminal device 100.

Further, the terminal processor 106 of the terminal device 100 functions as a fatigue information processor 107A and the annunciator 108. Here, when the fatigue information processor 107A in the present embodiment receives the received signal from the health support device 10B, the fatigue information processor 107A transmits that received signal, the user information, and request information of requesting the fatigue information corresponding to the received signal to the server device 600.

The server device 600 is formed of a general computer such as a personal computer, and is coupled to the terminal device 100 so as to be able to communicate with each other.

As shown in FIG. 13, the server device 600 is provided with a server-side communication unit 601, a server-side storage 602, a server-side processor 603, and so on.

The server-side communication unit 601 communicates with a plurality of the terminal devices 100 via the Internet. It should be noted that it is possible to adopt a configuration capable of communicating with a plurality of the health support devices 10B via the Internet.

In the server-side storage 602, there are accumulated the received signals transmitted from the terminal devices 100. On this occasion, the user information, namely the information related to the body of the user such as the height, the weight, the age, the sex of the user, is recorded so as to be associated with the received signal.

The server-side processor 603 retrieves and then executes the variety of programs stored in the server-side storage 602 to thereby function as a user group selector 604, a model generator 605, a fatigue detector 606, and a fatigue information transmitter 607.

The user group selector 604 classifies each of the users into one of a plurality of user groups. Specifically, the user group selector 604 clusters the plurality of users who use the present system, and then divides the result into a plurality of groups based on the user information of each of the users. Then, when the user group selector 604 receives the request information from the terminal device 100, the user group selector 604 selects (identifies) the group to which the user transmitting the request information belongs, based on the user information of that user.

The model generator 605 generates the fatigue detection model in substantially the same manner as the model generator 577 in the second embodiment. Here, in the present embodiment, the fatigue detection model is generated for each of the groups. Specifically, the model generator 605 extracts the received signals of the users who belong to the target group from the plurality of received signals accumulated in the sever-side storage 602, and then generates the fatigue detection model based on the plurality of received signals thus extracted.

Similarly to the fatigue detector 576A in the second embodiment, the fatigue detector 606 obtains the fatigue information using the fatigue detection model.

Specifically, the fatigue detector 606 inputs the received signals to the fatigue detection model corresponding to the group selected by the user group selector 604 to obtain the fatigue information.

The fatigue information transmitter 607 transmits the fatigue information obtained in the fatigue detector 606 to the terminal device 100.

In such a present embodiment, the fatigue detection device according to the present disclosure is constituted by the health support device 10B and the server device 600.

Then, a fatigue detection method and a health support method using the support system according to the present embodiment will be described.

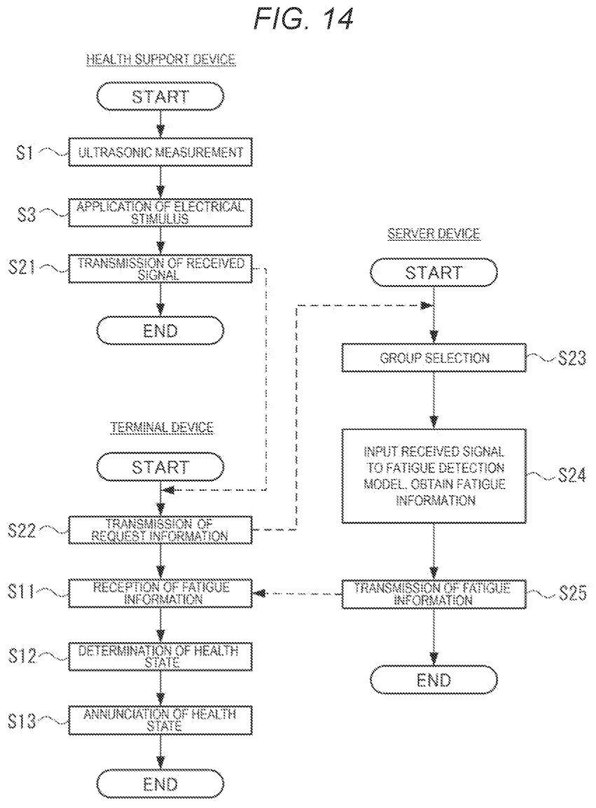

FIG. 14 is a flowchart showing the fatigue detection method and the health support method using the support system LA according to the present embodiment.

In the present embodiment, similarly to the first embodiment and the second embodiment, the step S1 is performed to perform the ultrasonic measurement by the ultrasonic device 20 to obtain the received signal, and then the step S3 is performed to apply the electrical stimulus to the muscle.

Similarly to the first embodiment and the second embodiment, the ultrasonic measurement in the step S1 and the application of the electrical stimulus to the muscle in the step S3 are each performed with a constant period. The period of the ultrasonic measurement in the step S1 is shorter than the period of the application of the electrical stimulus in the step S3, and the ultrasonic measurement is performed at least at the timing (the first time) when the electrical current is applied from the electrical stimulator 40 and the timing (the second time) when no electrical current is applied from the electrical stimulator 40.

Then, the health support device 10B transmits (step S21) the received signals thus obtained to the terminal device 100, and the terminal device 100 transmits (step S22) the request information including the received signals thus received and the user information to the server device 600.

When the user group selector 604 of the server device 600 receives the request information, the user group selector 604 selects (step S23) the group corresponding to the user based on the user information included in the request information. Then, the fatigue detector 606 inputs the received signals transmitted in the step S22 to the fatigue detection model corresponding to the group thus selected to thereby obtain (step S24) the fatigue information output from the fatigue detection model.

Subsequently, the fatigue information transmitter 607 transmits (step S25) the fatigue information obtained in the step S24 to the terminal device 100 which has transmitted the request information.

Thereafter, the processing in the step S11 through the step S13 similarly to the first embodiment is performed.

In the present embodiment, the received signals transmitted from the terminal devices 100 of the plurality of users are accumulated, and the fatigue detection model is generated for each of the groups clustered based on the user information.

The received signal when the user applies the external stimulus to the tissue such as a muscle using the electrical stimulator 40 of the health support device 10B is transmitted to the server device 600 together with the user information, then the fatigue detection model of the group corresponding to the user information is selected in the server device 600, and then the received signal is input to that fatigue detection model, and thus, the fatigue information is obtained.

In this case, it is possible even for the user who has just gotten the health support device 10B to perform the determination of the fatigue state high in accuracy by using the fatigue detection model generated based on the received signals of the users similar to that user.

### Modified Examples

The present disclosure is not limited to the embodiments described above, and includes the modifications described below within the range where the advantages of the present disclosure can be obtained.

### Modified Example 1

In the second embodiment, there is described the example in which the processor 57 of the health support device 10A functions as the model generator 577, and the fatigue detection model and the received signals are recorded on the memory 56, but this is not a limitation.

For example, the received signals when performing the ultrasonic measurement by the health support device 10A can be accumulated in the storage 105 of the terminal device 100.

In this case, it is possible for the terminal processor 106 to function as the model generator to generate the fatigue detection model based on the received signals accumulated in the storage 105. The fatigue detection model thus generated can be transmitted to the health support device 10A, and then stored in the memory 56, or can be stored in the storage 105. When recording the fatigue detection model on the storage 105, it is possible for the terminal processor 106 to be configured to function as the fatigue detector.

In such a configuration, the fatigue detection device according to the present disclosure is constituted by the health support device 10A and the terminal device 100.

### Modified Example 2

It is possible to combine the second embodiment and the third embodiment to switch the fatigue detection model in accordance with the elapsed time from when the user gets the health support device 10B, the number of times (the number of the received signals accumulated) of the ultrasonic measurement performed by the user, and so on.

For example, when only a short time has elapsed after the user bought the health support device 10B, the number of times of the ultrasonic measurement performed on the user is small, and the number of the received signals accumulated is small. Alternatively, when a predetermined period (e.g., three months or one year) has elapsed from the timing at which the previous ultrasonic measurement has performed, the reliability of the received signals thus accumulated decreases. In this case, as in the third embodiment, the fatigue detector 606 inputs the received signals obtained by the ultrasonic measurement to the fatigue detection model corresponding to the group to which the user belongs, to obtain the fatigue information.

In contrast, when the number of the received signals which are obtained in the latest predetermined period, and are accumulated is no smaller than a certain number, the fatigue detector 576A uses the fatigue detection model recorded on the memory 56 to obtain the fatigue information as in the second embodiment. It should be noted that the fatigue detection model having been personalized, as in the modified example 1, can be stored in the server-side storage 602 of the server 600, and in this case, the fatigue detector 606 inputs the received signals transmitted from the terminal device 100 to that fatigue detection model.

### Modified Example 3

In the second embodiment and the third embodiment described above, there is described the example of using the fatigue detection model taking the received signal as the input, and the fatigue information representing the fatigue state as the output. In contrast, it is possible to use the fatigue detection model taking the tissue thickness calculated by the thickness calculator 574 as the input, and the fatigue information as the output. Alternatively, it is also possible to use the fatigue detection model taking the change ratio of the tissue thickness detected by the tissue state detector 575 as the input, and the fatigue information as the output.

### Modified Example 4

In the first embodiment, there is described the example in which the fatigue detector 576 compares the change ratio of the tissue thickness detected by the tissue state detector 575 and the predetermined threshold value with each other to detect the fatigue state. Here, in the first embodiment, there is described the example in which the value set in advance based on the user information is used as the threshold value.

In contrast, it is possible to adopt a configuration in which the received signals are accumulated, and the threshold value is updated based on these received signals.

### Modified Example 5

In the first embodiment, there is described the example in which the tissue state detector 575 detects the change ratio of the tissue thickness, but it is possible to arrange that the change amount of the tissue thickness is detected.

### Modified Example 6

In the embodiments described above, there are illustrated the health support devices 10, 10A, and 10B which apply the electrical stimulus to the muscle with the electrical stimulator 40 to thereby expand and contract the muscle, and then measure the muscle thickness, but it is not required to dispose the electrical stimulator 40 as described above.

It is possible for the user to voluntarily exercise to thereby expand and contract the muscle, and measure the change in the muscle thickness on that occasion with the ultrasonic device 20. Alternatively, it is possible to apply other external stimuli than the electrical stimulus to the muscle.

Further, the target tissue is not limited to the muscle, and it is possible to assume an internal organ such as a stomach, or a blood vessel as the target. When assuming the stomach as the target, it is possible to determine the fatigue state of the stomach by measuring expansion and contraction of the stomach after eating with the ultrasonic device 20, and thus, determine the health state related to digestion. Further, when assuming the blood vessel as the target, it is possible to determine the fatigue (aging) of the blood vessel to determine the health state such as arteriosclerosis.

### Conclusion of Present Disclosure

A fatigue detection device according to a first aspect of the present disclosure includes an ultrasonic device configured to transmit an ultrasonic wave into a living body, then receive the ultrasonic wave reflected by a tissue in the living body to output a received signal, and at least one processor, wherein the processor obtains the received signal output from the ultrasonic device, and detects a fatigue state of the tissue from a change in the received signal when a load is applied to the living body.

Thus, it is possible to detect the fatigue state of the tissue using the non-invasive method, and it is possible even for an ordinary user who does not have medical expertise to easily confirm the fatigue state of him- or herself. Further, for example, since it is possible to detect the state such as the thickness of the tissue by the ultrasonic measurement, it is possible to more accurately detect the fatigue state of the tissue to be the target compared to, for example, when measuring an amount of a component related to the fatigue such as a lactic acid level in the blood, or when measuring the state of the swollenness to thereby detect the fatigue deriving from the swollenness.

In the fatigue detection device according to the present aspect, the processor may include a tissue state detector configured to detect a change in the tissue from the received signal, and a fatigue detector configured to detect a fatigue state of the tissue based on the change in the tissue detected by the tissue state detector.

In the present aspect, since it is possible to directly detect the change in tissue based on the received signal obtained by the ultrasonic measurement by the ultrasonic device, it becomes possible to accurately detect the fatigue state based on the change in that tissue.

In the fatigue detection device according to the present aspect, the change in the tissue may be a change ratio of a thickness of the tissue when a load is applied to the tissue.

Thus, it is possible to reduce an error due to an individual difference of the users to increase the accuracy of the fatigue detection.

In the fatigue detection device according to the present aspect, there may be adopted a configuration in which the received signal includes a first received signal obtained in a first time, and a second received signal obtained in a second time different from the first time, and the processor inputs the first received signal and the second received signal to a machine learning model which takes the first received signal and the second received signal as input, and takes a fatigue state of the tissue as output to obtain the fatigue state.

In such a configuration, by inputting the received signals to the machine learning model, it is possible to detect the fatigue state of the tissue. Therefore, it is possible to achieve the simplification of the configuration and speeding up of the processing.

Further, by accumulating the received signals of the respective users, it is possible to increase the accuracy of the machine learning model, and it is possible to perform the detection of the fatigue state higher in accuracy using the highly personalized machine learning model.

A support system according to a second aspect of the present disclosure is a support system including the fatigue detection device described above, a fatigue information processor, and an annunciator, wherein the fatigue detection device outputs fatigue information including information related to a fatigue state to the fatigue information processor, the fatigue information processor determines a health state of a user based on the fatigue information, and the annunciator informs the user of information related to the health state determined by the fatigue information processor.

Thus, it is possible to accurately detect the fatigue state with respect to the tissue to be the target as described above, and it is possible to appropriately determine the health state of the user in accordance with that fatigue state and announce the health state.

A fatigue detection method according to a third aspect of the present disclosure is a fatigue detection method of detecting a fatigue state of a tissue using an ultrasonic device configured to transmit an ultrasonic wave into a living body, and receive the ultrasonic wave reflected by the tissue in the living body, the method including outputting a received signal corresponding to the ultrasonic wave which is transmitted into the living body from the ultrasonic device, then reflected by the tissue, and then received by the ultrasonic device, and detecting a fatigue state of the tissue from a change in the received signal when a load is applied to the living body.

Thus, it is possible to obtain substantially the same functions and advantages as those of the first aspect.

What is claimed is:

1. A fatigue detection device comprising:
an ultrasonic device configured to transmit an ultrasonic wave into a living body, then receive the ultrasonic wave reflected by a tissue in the living body to output a received signal; and
at least one processor, wherein
the processor
obtains the received signal output from the ultrasonic device, and
detects a fatigue state of the tissue from a change in the received signal when a load is applied to the living body.

2. The fatigue detection device according to claim 1, wherein
the processor includes
a tissue state detector configured to detect a change in the tissue from the received signal, and
a fatigue detector configured to detect a fatigue state of the tissue based on the change in the tissue detected by the tissue state detector.

3. The fatigue detection device according to claim 2, wherein
the change in the tissue is a change ratio of a thickness of the tissue when a load is applied to the tissue.

4. The fatigue detection device according to claim 1, wherein
the received signal includes a first received signal obtained in a first time, and a second received signal obtained in a second time different from the first time, and
the processor inputs the first received signal and the second received signal to a machine learning model which takes the first received signal and the second received signal as input, and takes a fatigue state of the tissue as output to obtain the fatigue state.

5. The fatigue detection device according to claim 3, wherein
the received signal includes a first received signal obtained in a first time, and a second received signal obtained in a second time different from the first time, and
the processor inputs the first received signal and the second received signal to a machine learning model which takes the first received signal and the second received signal as input, and takes a fatigue state of the tissue as output to obtain the fatigue state.

6. A support system comprising:
the fatigue detection device according to claim 1;
a fatigue information processor; and
an annunciator, wherein
the fatigue detection device outputs fatigue information including information related to a fatigue state to the fatigue information processor,
the fatigue information processor determines a health state of a user based on the fatigue information, and
the annunciator informs the user of information related to the health state determined by the fatigue information processor.

7. A support system comprising:
the fatigue detection device according to claim 5;
a fatigue information processor; and
an annunciator, wherein
the fatigue detection device outputs fatigue information including information related to a fatigue state to the fatigue information processor,
the fatigue information processor determines a health state of a user based on the fatigue information, and
the annunciator informs the user of information related to the health state determined by the fatigue information processor.

8. A fatigue detection method of detecting a fatigue state of a tissue using an ultrasonic device configured to transmit an ultrasonic wave into a living body, and receive the ultrasonic wave reflected by the tissue in the living body, the method comprising:
outputting a received signal corresponding to the ultrasonic wave which is transmitted into the living body from the ultrasonic device, then reflected by the tissue, and then received by the ultrasonic device; and
detecting a fatigue state of the tissue from a change in the received signal when a load is applied to the living body.
